(19) United States
(12) Patent Application Publication
(10) Pub. No.: US 2023/0264955 A1
(43) Pub. Date: Aug. 24, 2023

(54) PROCESS FOR PRODUCING A GAS STREAM COMPRISING CARBON MONOXIDE

(71) Applicant: JOHNSON MATTHEY DAVY TECHNOLOGIES LIMITED, London (GB)

(72) Inventors: Henry Arthur CLAXTON, London, Greater London (GB); Amelia Lorna Solveig COOK, London, Greater London (GB); Mark Joseph MCKENNA, Billingham, Cleveland (GB)

(21) Appl. No.: 18/006,681

(22) PCT Filed: Sep. 17, 2021
(86) PCT No.: PCT/GB2021/052422

§ 371 Date: Jan. 24, 2023

(30) Foreign Application Priority Data
Oct. 16, 2020 (GB) 2016417.4

(51) Int. Cl. C01B 3/48; C01B 3/50; B01J 19/24; C07C 1/04; B01D 53/26; B01D 53/14

(52) U.S. Cl. CPC C01B 3/48; C01B 3/506; B01J 19/245; C07C 1/043; B01D 53/265; B01D 53/1475; C01B 2203/0255; C01B 2203/0283; C01B 2203/046; C01B 2203/0495; C01B 2203/0475; C01B 2203/1058; C01B 2203/1082; C01B 2203/062; C07C 2523/75; B01D 2257/80; B01D 2257/504

(57) ABSTRACT

A process for producing a gas stream comprising carbon monoxide comprising the steps of (a) feeding a gas mixture comprising carbon dioxide and hydrogen to a burner and combusting it with a sub-stoichiometric amount of an oxygen gas stream to form a combusted gas mixture comprising carbon monoxide, carbon dioxide, hydrogen and steam, (b) passing the combusted gas mixture through a bed of reverse water-gas shift catalyst to form a crude product gas mixture containing carbon monoxide, steam, hydrogen and carbon dioxide, (c) cooling the crude product gas mixture to below the dew point and recovering a condensate to form a dewatered product gas, (d) removing carbon dioxide from the dewatered product gas in a carbon dioxide removal unit to form the gas stream comprising carbon monoxide, and (e) combining carbon dioxide recovered by the carbon dioxide removal unit with the gas mixture comprising hydrogen and carbon dioxide.

This invention relates to a process producing a gas stream comprising carbon monoxide from a feed gas comprising carbon dioxide and hydrogen by the reverse water-gas shift reaction.

Gas streams comprising carbon monoxide may be used in processes for the synthesis of various chemicals including hydrocarbons and oxygenates, such as alcohols.

The reverse water-gas shift reaction may be depicted as follows:

CO₂+H₂↔CO+H₂O ΔH==+9.8 kcal/mole

The reverse water-gas shift process is favoured at high temperatures.

WO2019175476A1 discloses a method for producing carbon monoxide by combining oxygen with a carbon dioxide stream to form a carbon dioxide-based mixture, combining the carbon dioxide-based mixture with a hydrogen based stream to form the gaseous feed, supplying a hydrocarbon containing stream to the hydrogen based stream before the supply of the carbon dioxide based mixture, and feeding the gaseous feed into a reactor that contains at least one catalyst. The gaseous feed is treated by means of a partial oxidation in the reactor such that carbon dioxide reacts with hydrogen in the reactor in presence of oxygen and heat is formed.

WO2020114899A1 discloses a process for performing the reverse water gas shift reaction at elevated temperature in a reaction vessel, wherein no catalyst is present in the reaction vessel, by introducing carbon dioxide, hydrogen and oxygen separately into the reaction vessel, where hydrogen and oxygen are introduced into the reaction vessel via a burner such that the temperature in the reaction vessel is maintained in the range of 1000 to 1500° C. by varying the molar ratio of hydrogen to oxygen. However, this process increases the amount of extra hydrogen that needs to be combusted within the process to close the heat balance.

We have found an improved method for more efficiently performing the reverse water-gas shift reaction with satisfactory conversion to produce a gas stream containing carbon monoxide.

Accordingly, the invention provides a process for producing a gas stream comprising carbon monoxide comprising the steps of (a) feeding a gas mixture comprising carbon dioxide and hydrogen to a burner disposed in a reverse water-gas shift vessel and combusting it with a sub-stoichiometric amount of an oxygen gas stream to form a combusted gas mixture comprising carbon monoxide, carbon dioxide, hydrogen and steam, (b) passing the combusted gas mixture though a bed of reverse water-gas shift catalyst disposed within the reverse water-gas shift vessel to form a crude product gas mixture containing carbon monoxide, steam, hydrogen and carbon dioxide, (c) cooling the crude product gas mixture to below the dew point and recovering a condensate to form a dewatered product gas, (d) removing carbon dioxide from the dewatered product gas in a carbon dioxide removal unit to form the gas stream comprising carbon monoxide, and (e) combining carbon dioxide recovered by the carbon dioxide removal unit with the gas mixture comprising hydrogen and carbon dioxide fed to the reverse water-gas shift vessel.

The invention further provides a system for producing a gas stream comprising carbon monoxide by the process.

In the process, a carbon dioxide stream and a hydrogen stream are combined to form a feed gas mixture. If desired, a portion of the hydrogen may be fed separately to the reverse water-gas shift vessel. If desired, a portion of the carbon dioxide may be fed separately to the reverse water-gas shift vessel.

Hydrogen is combusted in the reverse water-gas shift vessel to generate heat for the reverse water-gas shift reaction. Accordingly, hydrogen should be provided in excess of the carbon dioxide so that sufficient hydrogen remains after combustion to drive the reaction forward over the reverse water-gas shift catalyst. Excess hydrogen is also desirable in view of the potential end use of the carbon monoxide-containing gas in the Fischer-Tropsch synthesis of hydrocarbons where the H₂:CO ratio is desirably about 2:1. The molar ratio of hydrogen to carbon dioxide in the gas mixture fed to the burner, including the recycled carbon dioxide, may be in the range of 1:1 to 5:1. The ratio may vary depending on the conversion of the carbon dioxide achieved in the reverse water-gas shift unit and the desired hydrogen to carbon monoxide ratio for the downstream process.

The gas mixture comprising carbon dioxide and hydrogen fed to the burner, including the carbon dioxide recovered in step (d), may comprise 15 to 50% by volume, preferably 25 to 40% by volume, of carbon dioxide. The gas mixture comprising carbon dioxide and hydrogen fed to the burner preferably comprises less than 10% vol in total of other gases, such as steam, nitrogen, carbon monoxide and methane.

Any suitable source of carbon dioxide may be used. Thus, the carbon dioxide stream may be a stream recovered from a conventional ammonia plant that uses a hydrocarbon or carbonaceous feed, or the carbon dioxide stream may be one recovered from a furnace or boiler flue gas, wherein the furnace or boiler is heated by combustion of a fossil fuel, such as natural gas or coal, biomass, or carbonaceous wastes, such as plastics. Alternatively, the carbon dioxide may be a stream separated from air or seawater.

The gas mixture comprising hydrogen and carbon dioxide further contains at least a portion of the recovered carbon dioxide obtained from the carbon dioxide removal unit.

Any suitable source of hydrogen may be used. More than one source of hydrogen may be used. The process preferably utilises non-fossil fuel based hydrogen. Accordingly, the hydrogen may be generated by catalytic or non-catalytic partial oxidation of biomass or plastics, optionally followed by steam reforming of the partial oxidation product gases. Alternatively. the hydrogen may be provided by splitting water. Preferably, the hydrogen is electrolytic hydrogen, for example hydrogen formed by the electrolysis of water. Intermediate storage of the hydrogen may be used to reduce any variability in production of hydrogen from the electrolysis.

Any suitable source of oxygen may be used. The oxygen purity may be at least 94% by volume, preferably at least 98% by volume or 99% by volume to minimise inerts such as nitrogen in the carbon monoxide product stream. The oxygen need not be combined with a carbon dioxide stream, unlike WO2019175476. Oxygen may be recovered from air using an air separation unit (ASU), which may be driven by renewable power sources or steam raised in the reformed gas boiler or other sources, including from downstream processes. Preferably, the oxygen comprises electrolytic oxygen, for example oxygen formed by the electrolysis of water. If desired, steam may be included with the oxygen.

Hydrogen and oxygen for the process are therefore both preferably generated using an electrolysis unit to which a source of water is fed. The water may include condensate recovered from the crude product gas mixture, and/or may comprise the water recovered from a downstream conversion unit such as a Fischer-Tropsch hydrocarbon synthesis unit. If required, the water may be treated to remove contaminants, such as organic compounds or salts, that would adversely affect the electrolysis unit.

The electricity for the electrolysis unit is desirably not obtained from the combustion of fossil fuels. The electrical power for the electrolysis may be provided by nuclear power or preferably, by renewable power sources, such as photovoltaic solar energy, wind energy, tidal energy, waterpower or hydroelectricity, marine energy sources, geothermal energy and/or biomass. The electricity for the electrolysis may also be provided using a turbine driven by steam generated using heat recovered from product gas streams created by the partial oxidation of biomass or plastic waste. Electrical power may be stored in an intermediate facility such pumped hydro- or battery-storage to provide a more constant supply of electrical power to the electrolysis unit.

The electrolysis unit typically comprises one or more electrolysers that operates according to the general formula:

Electricity+2H₂O→2H₂+O₂

Electrolysis is the process for chemical decomposition of water to give oxygen and hydrogen under the action of an electric current. In one arrangement, alkaline cell electrolysis may be used in the process. Alkaline cell electrolysis may be performed at temperatures below 200° C. by combining water with potassium hydroxide, the concentration of which may vary as a function of the temperature (typically from 25% by weight at 80° C. up to 40% by weight at 160° C.). Potassium hydroxide is preferred to sodium hydroxide, essentially for reasons of superior conductivity at an equivalent temperature level. Alternatively, polymer-electrode membrane electrolysers may be used. Alternatively, high-temperature electrolysis may be used in the process. High-temperature electrolysis is operated at high temperature (700 to 900° C.) and at reduced pressure. High-temperature electrolysis is more efficient than the process at ambient temperature since a portion of the energy necessary for the reaction is contributed via the heat, which is often cheaper to obtain than electricity, and electrolysis reactions have a better yield at high temperature. High temperature electrolysis may also enable conversion of carbon dioxide in the water to carbon monoxide. The carbon monoxide may advantageously be used to supplement the syngas being fed to the downstream FT unit.

The carbon dioxide and hydrogen streams or the gas mixture comprising the carbon dioxide and hydrogen may, if required, be compressed to a pressure in the range of 0.8 to 4 MPa or, optionally, 5 Mpa (gauge), preferably 1.2 to 3.2 MPag.

The oxygen stream is desirably provided at a pressure above that of the gas mixture fed to the burner, for example up to 8 bar above that of the gas mixture fed to the burner, because this generates a differential velocity and promotes mixing in the burner flame. The oxygen stream may be pre-heated if desired to improve combustion.

Before, but preferably after compression, the gas streams fed to the reverse water-gas shift vessel may be preheated. The pre-heat temperature of the feed gases to the reverse water-gas shift vessel may be in the range of 400 to 1000° C. or 450 to 800° C. to sustain combustion. The hydrogen and carbon dioxide streams may be premixed before preheating or preheated and mixed. Preheating of the feeds to their pre-heat temperatures may be done by interchange with the crude product gas mixture, and/or by steam heating, or by using a fired heater or by electrical heating or by a combination of two or more these. Preferably, the feed gas mixture comprising carbon dioxide and hydrogen is heated by interchange with the crude product gas mixture.

While generally it is preferable to minimise the steam fed to the reverse water-gas shift vessel, it may be advantageous to include steam in the oxygen gas stream, especially during start-up or shut-down of the process in order to safely transition between the phases of operation. The amount of steam in the oxygen stream may be in the range of 0 to 50% by volume.

The amount of oxygen fed to the burner is sub-stoichiometric, i.e. the amount of oxygen is insufficient to combust all of the hydrogen in the gas mixture. The combustion of hydrogen consumes two hydrogen molecules for every molecule of oxygen. The molar ratio of oxygen to hydrogen (O₂:H₂) is therefore typically less than 0.5:1 and may be in the range 0.02 to 0.2:1 or 0.05 to 0.15:1.

The oxygen and the gas mixture comprising carbon dioxide and hydrogen are fed to a burner disposed in a reverse water-gas shift vessel. Any burner design may be used, such as burners used in autothermal or secondary steam reformers. The streams may be fed at a single point or at multiple points. Burner designs where the gas mixture is fed to a neck region of the reverse water-gas shift vessel and the oxygen is fed to a central conduit passing though the neck region and opening into a combustion zone are preferred. Combustion generates a flame in a combustion zone upstream of the water-gas shift catalyst within the reverse water-gas shift vessel. The localized conditions in the combustion section, especially in the flame front region, may be controlled by managing the momentum of the oxidant and gas streams. The water-gas shift vessel may be orientated such that the combustion zone is above the bed of reverse water-gas shift catalyst. Such arrangements are used in autothermal or secondary steam reforming vessels and may be used in the present process, which may be termed autothermal reverse water-gas shift (ARWGS). Other arrangements of the burner and catalyst may however be used.

The reverse water-gas shift vessel comprises two reaction zones. The first zone, i.e. the combustion zone, is defined by the region between the burner and the inlet to the catalyst bed. The burner in the reverse water-gas shift vessel may be located in a neck region and discharge into a void space, for example in the shape of a frustum cone or cylinder, with a vertical axis. In this zone, the process gas and process oxidant mix together and the oxygen—which is present in less than stoichiometric ratio—is consumed. The second reaction zone is defined by the bed of reverse water-gas shift catalyst. This zone is typically cylindrical in shape, with the cylinder axis vertical. An objective in the design of the reverse water-gas shift vessel is to reduce variations of the temperature and composition of the process gas stream leaving the first reaction zone and entering the second reaction zone. Non-uniform conditions can lead to catalyst damage and/or loss of catalyst activity. In order to obtain a uniform gas mixture at the inlet to the catalyst, it is necessary to intimately mix the process gas with the process oxidant. In oxygen-based reactors, the mass flowrate of the oxidant is much less than that of the process gas. Dispersing a relatively smaller flowrate of oxidant into a relatively larger flowrate of process gas requires the oxidant to be accelerated to higher velocity. The preferred approach is to employ a burner mounted in a cylindrical neck region of the vessel, above the combustion zone referred to above. The dimensions of the burner and neck are selected to stabilise the flame on the burner and to enhance mixing between the streams of process oxidant and process gas. The gas mixture is heated by the combustion to a temperature typically in the range of 800 to 1300° C. Oxygen is consumed in the combustion zone. The heated gas mixture comprising carbon monoxide, carbon dioxide, steam, and unreacted hydrogen is then passed through a bed of reverse water-gas shift catalyst disposed within the reverse water-gas shift vessel downstream of the burner.

The reverse water-gas shift catalyst may be any suitable transition metal oxide catalyst, for example a catalyst based on nickel oxide, iron oxide or on chromium oxide, but other catalysts used as reverse water-gas shift catalysts may be used. Preferably the catalyst is a nickel-oxide based catalyst. Such catalysts are active for the reverse water-gas shift catalyst but advantageously will also steam reform hydrocarbons that may be present in the feed gas mixture. The catalyst therefore preferably comprises nickel oxide on a suitable refractory metal oxide support. The refractory metal oxide support may comprise zirconia, alumina, calcium aluminate, magnesium aluminate, titania magnesia, or mixtures thereof. More preferably, the catalyst comprises nickel oxide on zirconia, nickel oxide on alpha-alumina, nickel oxide on calcium aluminate or nickel oxide on magnesium aluminate. The nickel content may be in the range 3 to 20% by weight, expressed as NiO.

The reverse water-gas shift catalyst may be particulate, for example in the form of shaped units such as pellets, rings or extrudates, which may be lobed or fluted. The catalytically active metal, e.g. nickel, may be dispersed throughout the particulate catalyst or present only within an eggshell layer of thickness 200 to 1000 micrometres on the surface of the refractory support. Alternatively, catalyst may comprise one or more monolithic supports such as a metal or ceramic foam or honeycomb supporting the catalytically active metal. Preferably, the catalyst is a particulate catalyst, more preferably 4-hole cylinder, particularly one that is a lobed or fluted to provide a higher geometric surface area (GSA) than a similarly sized solid cylinder without increasing pressure drop. Catalysts having a GSA in the range 400-550 m² per cubic metre are preferred.

If desired, a layer of zirconia balls, pellets or tiles may be placed on top of the catalyst to protect the surface of the catalyst from irregularities in the combusting gas flow. A benefit of providing this layer is to prevent disturbance of the surface of the catalyst bed.

By controlling the pre-heat temperature and the amount of oxygen fed to the burner, it is possible to control the exit temperature of the reverse water-gas shift vessel. The exit temperature may be in the range 700° C. to 1050° C., preferably 750 to 950° C.

In addition to producing the carbon monoxide gas stream by the reverse water-gas shift reaction, the reverse water-gas shift vessel with an appropriate selection of catalyst may also be used to convert waste gases from downstream processes into carbon monoxide. The reverse water-gas shift vessel may therefore also be fed with hydrocarbon or oxygenates or, preferably, a pre-reformed gas mixture derived from hydrocarbon or oxygenates that do not contain hydrocarbons higher than methane. The use of pre-reformed gas mixtures is preferred because it reduces the risk of unwanted carbon formation in the reverse water-gas shift vessel or on the reverse water-gas shift catalyst.

Pre-reforming may be performed by passing a feed gas comprising the hydrocarbon- or oxygenate-containing gas stream, mixed with an appropriate amount of steam, through a pre-reformer vessel containing a fixed bed of pre-reforming catalyst. The steam introduction may be effected by direct injection of steam and/or by saturation of the feed gas by contact with a stream of heated water. The heated water may comprise condensed water from a downstream process that contains soluble organic compounds. Alternatively, the steam used for direct injection may have been used to strip organic compounds from condensed water from a downstream process. In this way, the organic compounds may be converted to hydrogen and carbon oxides in the pre-reformer and the burden of waste water treatment for the downstream process may be reduced. The amount of steam introduced may be such as to give a steam to carbon ratio of 1:1 to 5:1, preferably 1:1 to 3:1, i.e. 1 to 3 moles of steam per mole of carbon atoms contained in hydrocarbons in the pre-reformer feed gas. The pre-reformer feed gas, typically at an inlet temperature in the range of 350-650° C., more suitably 350-500° C., may be passed adiabatically through a bed of a steam reforming catalyst, such as a nickel steam reforming catalyst having a high nickel content, for example above 40% by weight. During the adiabatic pre-reforming step, any hydrocarbons higher than methane react with steam to give a mixture of methane, carbon oxides and hydrogen.

The gas mixture comprising hydrogen and carbon monoxide may be combined with the hydrocarbon- or oxygenate-containing stream, or the pre-reformed gas stream, and preheated upstream of the burner. Alternatively, the hydrocarbon- or oxygenate-containing stream, or the pre-reformed gas stream may be preheated and fed separately to the burner.

In some embodiments, the reverse water-gas shift vessel may be fed with a gas mixture comprising methane and carbon dioxide formed by pre-reforming a Fischer-Tropsch tail gas and optionally non-condensable hydrocarbons recovered from a downstream Fischer-Tropsch process, such as from a Fischer-Tropsch products upgrading unit, such as a hydrocracker.

The crude product gas mixture from the reverse water-gas shift vessel comprises steam formed by the reverse water-gas shift reaction and possibly steam added with the feed gases.

Water is recovered from the crude product gas mixture by cooling the product gas mixture to below the dew point and separating condensate, e.g. using one or more conventional gas-liquid separators. Removing water condensate from the crude product gas mixture produces a dewatered product gas. The cooling may be performed by raising steam and/or by preheating one or more of the hydrogen stream, the carbon dioxide stream, the mixed gas stream comprising hydrogen and carbon dioxide, and optionally the pre-reformer feed gas and the pre-reformer effluent, where present. Further cooling with cold water and/or air may also be performed. Process steam generated by the cooling may be used in the pre-reforming step or in downstream processes and/or for power generation.

The condensed water may, if desired, be recycled at least in part to the process. The condensate may be used, after treatment if desired, be used as boiler feed water. In addition, or alternatively, the condensate, optionally after treatment to use contaminants, may be fed to an electrolysis unit used to generate hydrogen for the process. Accordingly, in some embodiments, a water stream recovered from the crude product gas mixture may be fed to an electrolysis unit. Condensate may also be used, again after treatment if desired, as a boiler feed water.

The crude product gas mixture contains carbon dioxide, which is removed from the dewatered product gas using a carbon dioxide removal unit. The majority of the carbon dioxide may be separated by membrane, solid absorbent or, preferably, a wash system, such as a system operating by counter current contact of the crude product gas mixture or dewatered product gas with absorbent liquid over packing in a tower. The absorbent liquid can be a physical solvent such as potassium carbonate (sold as the Benfield process), methanol (sold as the Rectisol process) or glycols (sold as the Selexol process) or chemical solvents such as amines. The carbon dioxide removal unit may therefore include one or more vessels providing a physical wash system or a reactive wash system, preferably a reactive wash system, especially an amine wash system. The carbon dioxide may be removed by a conventional acid gas recovery unit (AGRU). In a conventional AGRU, a de-watered gas stream is contacted with a stream of a suitable absorbent liquid, such as an amine, for example an aqueous solution comprising monoethanolamine (MEA), methyldiethanolamine (MDEA) or dimethylethanolamine (DMEA), particularly methyl diethanolamine (MDEA), so that the carbon dioxide is absorbed by the liquid to give a laden absorbent liquid and a gas stream having a decreased content of carbon dioxide. The laden absorbent liquid is then regenerated by heating and/or reducing the pressure to desorb the carbon dioxide and to give a regenerated absorbent liquid, which is then recycled to the carbon dioxide absorption stage. Heat from the regeneration of the laden absorbent may be recovered from within the process. For example, a portion of the crude product gas mixture or steam generated by cooling the crude product gas mixture may be used to heat the laden absorbent.

Alternatively, in place of the washing with amines, cold methanol or a glycol may be used in a similar manner as the amine to remove the carbon dioxide.

The recovered carbon dioxide obtained from the carbon dioxide removal unit is preferably recompressed as required and returned to the reverse water-gas shift vessel to increase the overall conversion to carbon monoxide.

The recovered carbon dioxide may be combined with the carbon dioxide feed, the hydrogen gas feed or the gas mixture containing hydrogen and carbon monoxide before pre-heating. It is preferably combined with the carbon dioxide feed stream before compression thereof.

The removal of carbon dioxide from the dewatered product gas produces a gas stream comprising carbon monoxide. Hydrogen will also be present in the product gas with the amount depending on the excess of hydrogen fed to the reverse water-gas shift vessel. Small amounts of carbon dioxide, methane and inert gases, such as nitrogen may also be present, but this is undesirable to prevent their build up in downstream processes, especially where the product gas is to be used for the production of Fischer Tropsch hydrocarbons. Furthermore, small amounts of catalyst poisons such as ammonia, hydrogen cyanide and sulphur compounds such as hydrogen sulphide may also be present Accordingly, one or more purification units may be provided downstream of the carbon dioxide removal unit.

The gas stream comprising carbon monoxide from the current process comprises carbon monoxide and hydrogen. The hydrogen to carbon monoxide molar ratio may be in the range 1.0 to 2.5:1, preferably 1.2 to 2.5:1, more preferably 1.6 to 2.2, which is particularly suitable for hydrocarbon synthesis by the Fischer-Tropsch reaction.

In a preferred use, the product gas is fed to a Fischer-Tropsch hydrocarbon synthesis unit that synthesizes a mixture of hydrocarbon products.

The Fischer-Tropsch hydrocarbon synthesis unit may comprise one or more Fischer-Tropsch reaction vessels containing a Fischer-Tropsch catalyst. The Fischer-Tropsch conversion stage can be carried out according to any one of the known processes, using any one of the known catalysts, but is advantageously applied to processes using cobalt catalysts.

The Fischer-Tropsch process involves a series of chemical reactions that produce a variety of hydrocarbons, ideally having the formula (CₙH₂ₙ₊₂). The more useful reactions produce alkanes as follows:

(2n+1)H₂+nCO→CₙH₂ₙ₊₂+nH₂O,

where n is typically 5-100 or higher, with preferred products having n in the range 10-20.

Typically, a portion of the carbon monoxide is converted in the one or more Fischer-Tropsch reactors to produce liquid hydrocarbon products and water, and a gaseous mixture containing unreacted hydrogen and carbon monoxide, plus carbon dioxide and gaseous light hydrocarbons including methane, ethane, propanes and butanes. The reaction product mixture may be cooled, and the aqueous and liquid hydrocarbon streams separated from the gas mixture using one or more gas-liquid separators. Optionally, the cooling may be such that propane and butane are also condensed and removed as liquids at this stage. The co-produced water may be separated using known hydrocarbon-water separators. In some embodiments, the water co-produced in the Fischer-Tropsch hydrocarbon synthesis unit may be treated to remove organic compounds and used in the process. For example, steam may be used to strip a portion of the co-produced water of organic compounds and the stripped water may, after optional additional purification, be used as feed to the electrolysis unit. Alternatively, the co-produced water may be treated to remove organic compounds and fed to a boiler to create steam for the process. The separated gas mixture, which may be termed “tail gas”, may be used in a number of ways. Preferably a first portion of the tail gas is recycled to the one or more Fischer-Tropsch reactors in a synthesis loop to increase the overall conversion of carbon monoxide to hydrocarbons. The fraction that is recycled to form the loop may be set to control the build-up of inert gases, such as methane, in the Fischer-Tropsch hydrocarbon synthesis unit to an acceptable level. The remaining portion still contains a valuable source of carbon. Accordingly, in some embodiments, a second portion of the tail gas may be recycled to the reverse water-gas shift unit. If desired, unwanted hydrocarbons produced in the Fischer-Tropsch process may be recycled to the process by mixing them with the tail gas fed to the reverse water gas shift unit. Preferably, the recycle to the reverse water-gas shift unit is via a steam reformer, preferably an adiabatic steam reformer or “pre-reformer”, that converts ethane and any higher hydrocarbons present in the second portion of the tail gas to methane. Steam may be added to the second portion to provide a suitable steam to carbon ratio for the steam reforming step. The portion that is not recycled to the reverse water-gas shift unit, which may be termed “purge gas”, is removed from the process to prevent the build-up of inert gases. This may be before or after the steam reforming step, if present.

The purge gas may optionally be treated to separate a stream enriched in inert components or depleted in carbon-containing components, for example by passing the purge gas through a membrane which is more permeable to the inert gases than carbon-containing components, or by chilling the purge stream and condensing out condensable substances, or using a solid absorbent, such as a zeolite.

The purge gas may be exported as fuel or used within the process in a fired heater or thermal oxidiser to heat feed to the reverse water-gas shift vessel or superheat steam. Preferably the purge gas is combusted as a fuel. If the purge gas is combusted, then a portion of the carbon dioxide in the resulting combustion or flue gas may be separated to reduce carbon dioxide emissions from the process. The carbon dioxide may be separated using the same method used to recover carbon dioxide from the reverse water-gas shift reactor product gas and optionally may share equipment such as a regenerator column.

The invention is illustrated by reference to the accompanying drawing in which:

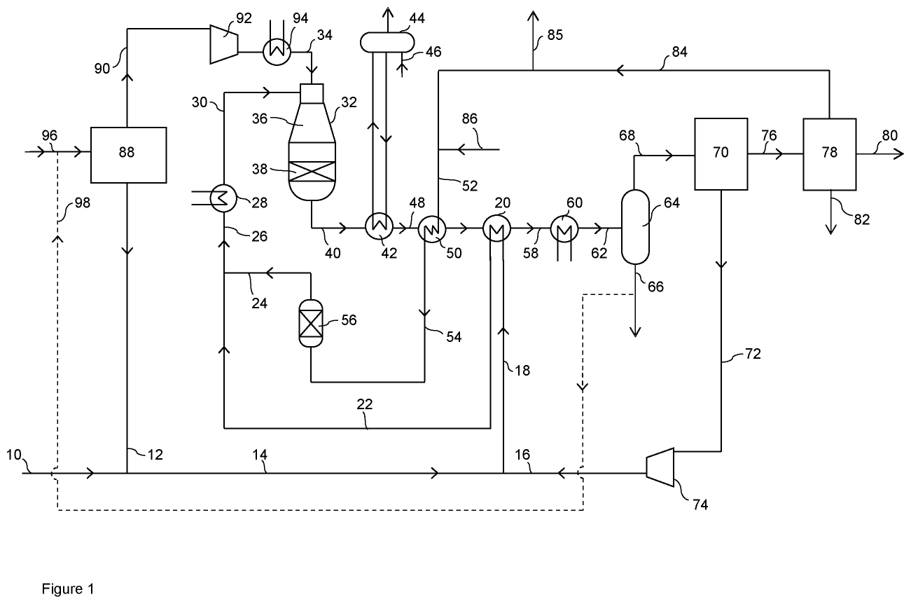

FIG. 1 is a diagrammatic flowsheet of one embodiment of the invention.

It will be understood by those skilled in the art that the drawings are diagrammatic and that further items of equipment such as reflux drums, compressors, pumps, vacuum pumps, towers, heat exchangers, temperature sensors, pressure sensors, pressure relief valves, control valves, flow controllers, level controllers, holding tanks, storage tanks, and the like may be required in a commercial plant. The provision of such ancillary items of equipment forms no part of the present invention and is in accordance with conventional chemical engineering practice.

In FIG. 1, a carbon dioxide stream, such as a carbon dioxide stream recovered from a flue gas, is fed to the process via line 10 and combined with a hydrogen stream provided by line 12 to form a mixed gas stream in line 14. A carbon dioxide recycle stream provided by line 16 is combined with the mixed gas in line 14 and the resulting mixed gas fed via line 18 to a gas-gas interchanger 20 where it is heated. The heated mixed gas is fed from the interchanger 20 via line 22 and combined with a pre-reformed tail gas mixture containing hydrogen, carbon dioxide, carbon dioxide, methane and steam, provided by line 24. The resulting mixed gas is provided via line 26 to a heater 28 where it is heated to the inlet temperature for the reverse water-gas shift reaction. Alternatively, the pre-reformed tail gas mixture 24 may be added downstream of the heater 28.

A heated feed gas mixture is passed from the heater 28 via line 30 to the inlet of a reverse water-gas shift vessel 32. The heated gas mixture is passed to the top of the vessel 32. A burner (not shown) located at the top of the vessel 32 receives a compressed and heated oxygen gas stream 34. The mixed gas and the oxygen combust at the inlet temperature, resulting in combustion of a portion of the hydrogen in a flame within a combustion zone 36 adjacent the burner within the vessel 32. The vessel 32 further comprises a bed of refractory metal oxide-supported nickel oxide reverse water-gas shift catalyst 38 disposed beneath the combustion zone 36. The catalyst promotes the reverse water gas shift reaction thereby forming carbon monoxide. The catalyst also steam-reforms methane in the pre-reformed tail gas from line 24 to form hydrogen and carbon oxides.

The resulting crude product gas mixture is recovered from the vessel 32 via line 40 and subjected to cooling in a boiler 42, connected to steam drum 44, fed with water via line 46. The partially cooled crude product is fed from the boiler 42 via line 48 to a heat exchanger 50 where it heats a mixture of Fischer-Tropsch tail gas and steam provided by line 52. The heated mixture is passed from the heat exchanger 50 via a line 54 to a pre-reformer vessel 56 containing a bed of nickel pre-reforming catalyst, to form the pre-reformed tail gas mixture 24. The crude product gas mixture is further cooled in heat exchanger 50. From heat exchanger 50, the partially cooled crude product gas is fed to interchanger 20 where it heats the feed gas mixture in line 18. From the interchanger 20 the partially cooled product gas is fed via line 58 to one or more further heat exchangers 60, which may be fed with cold water and/or air, where it is cooled to below the dew point to condense steam present in the crude product gas. A mixture of gas and condensate is passed from the one or more heat exchangers 60 via line 62 to a gas-liquid separator 64, where the condensate is separated and recovered via line 66.

A dewatered product gas comprising hydrogen, carbon monoxide and carbon dioxide is recovered via line 68 and fed to a conventional carbon dioxide removal unit 70, operating by means of a reactive liquid absorbent, that recovers carbon dioxide from the dewatered product gas. A carbon dioxide gas stream is recovered from the unit 70 via line 72 and compressed in compressor 74 to form the carbon dioxide recycle stream 16. A product gas mixture comprising carbon monoxide and hydrogen is recovered from the carbon dioxide removal unit 70 via line 76.

In this embodiment, the product gas comprising carbon monoxide in line 76 is subjected to one or more further steps of purification (not shown) and fed to a Fischer-Tropsch hydrocarbon synthesis unit 78 containing one or more Fischer-Tropsch reactors containing a cobalt Fischer-Tropsch hydrocarbon synthesis catalyst. The Fischer-Tropsch hydrocarbon synthesis unit converts the product gas into hydrocarbon products, which are recovered from the unit 78 via line 80. A co-produced water stream is recovered from the Fischer-Tropsch unit 78 via line 82. Within the unit 78, a Fischer-Tropsch tail gas stream is separated from the aqueous and liquid hydrocarbon streams. A portion of the tail gas stream comprising hydrogen, carbon monoxide, carbon dioxide, methane and higher hydrocarbons is recycled to the one or more Fischer-Tropsch reactors. A further portion of the Fischer-Tropsch tail gas stream is recovered from the unit 78 via line 84 and combined with steam provided by line 86 to form the mixture of Fischer-Tropsch tail gas and steam in line 52 fed to the pre-reformer 56. A remaining portion of the tail gas is taken from line 84 as a purge gas 85.

In this embodiment, an electrolysis unit 90 is used to electrolyse water to form the hydrogen stream 12 and to provide an oxygen stream 90 that is compressed in compressor 92 and heated in heater 94 to form the oxygen stream 34 fed to the reverse water-gas shift vessel 32.

Water for the electrolysis is provided to the electrolysis unit 88 via line 96. This water may optionally be supplemented by at least a portion of the condensate 66 fed to the electrolysis unit 84 via the dotted line 98.

In addition, the steam provided in line 86 may be derived at least in part from the co-produced water 82 recovered from the Fischer-Tropsch hydrocarbon synthesis unit 78.

1. A process for producing a gas stream comprising carbon monoxide comprising the steps of (a) feeding a gas mixture comprising carbon dioxide and hydrogen to a burner disposed in a reverse water-gas shift vessel and combusting it with a sub-stoichiometric amount of an oxygen gas stream to form a combusted gas mixture comprising carbon monoxide, carbon dioxide, hydrogen and steam, (b) passing the combusted gas mixture though a bed of reverse water-gas shift catalyst disposed within the reverse water-gas shift vessel to form a crude product gas mixture containing carbon monoxide, steam, hydrogen and carbon dioxide, (c) cooling the crude product gas mixture to below the dew point and recovering a condensate to form a dewatered product gas, (d) removing carbon dioxide from the dewatered product gas in a carbon dioxide removal unit to form the gas stream comprising carbon monoxide, and (e) combining carbon dioxide recovered by the carbon dioxide removal unit with the gas mixture comprising hydrogen and carbon dioxide fed to the reverse water-gas shift vessel.

2. The process according to claim 1, wherein the gas mixture comprises carbon dioxide and hydrogen has a hydrogen to carbon dioxide molar ratio in the range of 2:1 to 10:1.

3. The process according to claim 1, wherein the gas mixture comprising carbon dioxide and hydrogen fed to the burner comprises 15 to 50% by volume, preferably 25 to 40% by volume, of carbon dioxide.

4. The process according to claim 1, wherein the carbon dioxide is obtained from an ammonia plant that uses a hydrocarbon or carbonaceous feed, or from a furnace or boiler flue gas, wherein the furnace or boiler is heated by combustion of a fossil fuel or carbonaceous wastes, or from air or seawater.

5. The process according to claim 1, wherein hydrogen and oxygen for the process are generated using an electrolysis unit to which a source of water is fed.

6. The process according to claim 1, wherein the reverse water-gas shift catalyst comprises 3 to 20% wt nickel, expressed as NiO, on a refractory metal oxide support.

7. The process according to claim 1, wherein the carbon dioxide removal unit comprises a physical wash system or a reactive wash system.

8. The process according to claim 5, wherein the condensate, optionally after treatment to remove contaminants, is fed to the electrolysis unit.

9. The process according to claim 1, wherein the product gas stream comprising carbon monoxide is fed to a Fischer-Tropsch hydrocarbon synthesis unit.

10. The process according to claim 9, wherein a gas mixture comprising methane and carbon dioxide formed by pre-reforming a Fischer-Tropsch tail gas and optionally non-condensable hydrocarbons recovered from a downstream Fischer-Tropsch process are fed to the reverse water-gas shift unit.

11. A process according to claim 10, wherein water co-produced in the Fischer-Tropsch hydrocarbon synthesis unit is treated to remove organic compounds and used in the process.

12. A system for producing a gas stream by the process of claim 1 comprising (b) a feed line for feeding a gas mixture comprising carbon dioxide and hydrogen, (b) a reverse water-gas shift vessel operatively connected to the feed line, said reverse water-gas shift vessel comprising (i) a burner operatively connected to a source of oxygen, (ii) a combustion zone adjacent the burner, (iii) a bed of reverse water-gas shift catalyst disposed downstream of the combustion zone, and (iv) and outlet for a crude product gas mixture containing carbon monoxide, steam and carbon dioxide, (c) means operatively connected to the reverse water-gas shift vessel for cooling the crude product gas mixture to below the dew point and recovering a condensate therefrom to form a dewatered product gas, (d) a carbon dioxide removal unit coupled to the means for cooling the crude product gas mixture and recovering the condensate to recover carbon dioxide from the dewatered product gas, and (e) a conduit that feeds at least a portion of the recovered carbon dioxide from the carbon dioxide removal unit to the feed line for feeding the gas mixture comprising carbon dioxide and hydrogen to the reverse water-gas shift vessel.

13. A process according to claim 1, wherein the gas mixture comprising carbon dioxide and hydrogen fed to the burner comprises 25 to 40% by volume, of carbon dioxide.
